(19) United States
(12) Patent Application Publication
(10) Pub. No.: US 2026/0216450 A1
(43) Pub. Date: Jul. 30, 2026

(54) Thin Wall Cannula

(71) Applicants: Becton Dickinson France, Le Pont-de-Claix (FR); Becton, Dickinson and Company, Franklin Lakes, NJ (US)

(72) Inventors: Sandra Leang, Seyssinet Pariset (FR); Brigitte Duinat, Grenoble (FR); Benoit Fremon, Saint Martin d'Uriage (FR); Alfred W. Prais, Hewitt, NJ (US)

(21) Appl. No.: 19/441,653

(22) Filed: Jan. 6, 2026

Related U.S. Application Data
(63) Continuation-in-part of application No. PCT/US24/35995, filed on Jun. 28, 2024.

(30) Foreign Application Priority Data
Jul. 6, 2023 (EP) 23183866.5
Jan. 30, 2024 (EP) 24305165.3

(51) Int. Cl. A61M 5/32; A61M 5/315

(52) U.S. Cl. CPC A61M 5/329; A61M 5/31525

(57) ABSTRACT

The present invention relates to syringe and needle configurations that facilitate subcutaneous injection of viscous drug formulations, including high-concentration biologics such as omalizumab, while maintaining a relatively small needle outer diameter and providing acceptable injection performance under reasonable forces. The device includes a needle having a length of 12.7 mm, a gauge of 27, a minimum internal diameter of 0.277 mm, and 5 bevels on its distal surface. Methods of treatment using these devices for subcutaneous administration of omalizumab are also provided.

### CROSS-REFERENCE TO RELATED APPLICATIONS

This application is a Bypass Continuation-in-Part Application of International Application No. PCT/US24/35995 filed Jun. 28, 2024, and claims priority to European Patent Application No. 23183866.5 entitled “Thin Wall Cannula” filed Jul. 6, 2023, and also claims priority to European Patent Application No. 24305165.3 entitled “Thin Wall Cannula” filed Jan. 30, 2024, the disclosures of which are hereby incorporated by reference in their entireties.

### BACKGROUND OF THE INVENTION

### Field of the Invention

The present application provides syringe and needle configurations that facilitate subcutaneous injection of viscous drug formulations, including high-concentration biologics such as omalizumab, while maintaining a relatively small needle outer diameter and providing acceptable injection performance under reasonable forces. The invention further relates to methods of treating a patient by administering such drug formulations using the disclosed syringe and needle configurations.

### Description of Related Art

Cannulas are a ubiquitous component of modern medical care. Cannulas are used for example, to pierce a patient's skin to create a vascular access site for the administration of medicaments and other fluids (e.g., saline), or to withdraw blood from the patient. To reduce patient discomfort and the risk of injury, the outer diameter of cannulas is desirably minimized. However, the lumen of the cannula must be of sufficient diameter to allow the medicament or other fluid to flow through the cannula at an adequate rate, whatever the viscosity of the injected drug. As such, it is desirable to produce cannula with thin sidewalls to minimize the outer diameter while maximizing the inner diameter.

There are many viscous drugs known in the art that are compatible with use of this invention. Viscous drugs include, by way of non-limiting example, biologic drug products (including monoclonal antibodies), high-concentration protein formulations, peptide formulations, and other injectable compositions whose viscosity may increase due to concentration, excipients, or temperature. Such drugs may be administered subcutaneously in humans and may present delivery challenges including increased injection force, extended injection time, and reduced patient comfort. In particular embodiments described herein, a viscous drug comprises omalizumab.

With respect to omalizumab, it is known in the art to treat one or more of the following conditions by administering omalizumab to a patient: allergic asthma; food allergy; chronic rhino sinusitis with nasal polyps; chronic spontaneous urticaria; nasal polyps; and moderate to severe persistent asthma in patients with a positive skin test or in vitro reactivity to a perennial aeroallergen and symptoms inadequately controlled with inhaled corticosteroids (as described in XOLAIR® (omalizumab) Prescribing Information, 03/2023 Revision, Genentech, Inc. and Novartis AG).

It is further known in the art to administer omalizumab subcutaneously using a syringe. It is also known in the art to provide omalizumab in an injectable formulation having a concentration of 150 mg/mL, and to administer a volume corresponding to a clinically appropriate dose, including volumes such as 2 mL, 1 mL, or 0.5 mL (Somerville et al., Immunogenicity and safety of omalizumab in prefilled syringes in patients with allergic (IgE-mediate) asthma, Current Medical Research & Opinion Vol. 30, No. 1, 2014, 59-66).

It is further known in the art that the 150 mg/ml omalizumab formulation is an aqueous solution of 150 mg/mL antibody with 42.1 mg/mL L-arginine hydrochloride, 1.37 mg/mL L-histidine, 2.34 mg/mL L-histidine hydrochloride monohydrate, and 0.4 mg/mL polysorbate 20 (as described in XOLAIR® (omalizumab) Prescribing Information, 03/2023 Revision, Genentech, Inc. and Novartis AG).

It is also known in the art that the apparent viscosity of the 150 mg/mL omalizumab formulation is 12 to 14 m·Pas or, when measured at a shear rate of 200 s⁻¹ is: 24.7=0.5 m·Pas at 5.0±0.2° C., 15.3±0.5 m·Pas at 15.0±0.2° C., 11.4=0.5 m·Pas at 25.0±0.2° C., or 7.2=1.2 mPa·s at 40.0±0.2° C. (Soane et al. US 2021/0113697).

It is further known in the art that injectable formulations may contain visible and/or subvisible particles and that particle levels may be controlled to meet quality standards (for example, as described in USP <788> Particulate Matter in Injections). Specifically, with respect to the 150 mg/mL omalizumab formulation, it is known in the art to contain 6000 or fewer particles of diameter >10 μm and/or 600 or fewer particles of diameter >25 μm (Dubey EP 3 808 777 A1).

It is also known in the art to provide: a) at least 76% main omalizumab charge variant measured by ion exchange chromatography after being filled with 150 mg/mL omalizumab solution stored at 5° C.±1° C.; or b) at least 77% main omalizumab charge variant measured by ion exchange chromatography after being filled with 150 mg/mL omalizumab solution and then stored at 5° C.±1° C. for 2.5 months (Scholz et al. (US 2019/0216925)).

It is also known in the art to provide the proportion of the first peak of the syringe measured by hydrophobic interaction chromatography is at least: a) 62% after filling with 150 mg/mL omalizumab solution stored at 5° C.±1° C.; or b) 62% after being filled with 150 mg/mL omalizumab solution and then stored at 5° C.±1° C. for 2.5 months (Scholz et al. (US 2019/0216925)).

### SUMMARY

Embodiments of the present disclosure are directed to a medical injection cannula configured for use with a syringe. The cannula includes a sidewall defining a lumen and having a proximal end and a distal end, and a beveled tip defining an opening to the lumen, wherein a length-to-diameter ratio of the cannula is defined by a ratio

$$\frac{{L}_{hydrodynamic theoretical needle}}{{D}_{needle \_ inner}^{4}} ,$$

wherein L_{hydrodynamic theoretical needle} is a theoretical hydrodynamic length of the cannula, and D_{needle_inner} is an inner diameter of the sidewall of the cannula, and wherein the length-to-diameter ratio is comprised between 7.00×10¹¹ and 9.40×10¹² m⁻³.

In accordance with an embodiment, wherein the theoretical hydrodynamic length of the cannula L_{hydrodynamic} extends from the proximal end of the sidewall to a proximal end of the opening of the beveled tip.

In accordance with an embodiment, the outer diameter of the sidewall corresponds to a cannula standard for a 27G, 29G or 30G cannula.

In accordance with an embodiment, the outer diameter of the sidewall is for the 27G cannula and the ratio

$$\frac{{L}_{hydrodynamic theoretical needle}}{{D}_{needle \_ inner}^{4}}$$

is comprised between 8.00×10¹¹ m⁻³ and 1.4×10¹² m⁻³.

In accordance with an embodiment, the outer diameter of the sidewall is for the 29G cannula and the ratio

$$\frac{{L}_{hydrodynamic theoretical needle}}{{D}_{needle \_ inner}^{4}}$$

is comprised between 2.50×10¹² m⁻³ and 4.10×10¹² m⁻³.

In accordance with an embodiment, the outer diameter of the sidewall is for the 30G cannula and the ratio.

$$\frac{{L}_{hydrodynamic theoretical needle}}{{D}_{needle \_ inner}^{4}}$$

is comprised between 4.90×10¹² m⁻³ and 8.20×10¹² m⁻³.

In accordance with an embodiment, the outer diameter of the sidewall is for the 27G cannula and the ratio

$$\frac{{L}_{hydrodynamic theoretical needle}}{{D}_{needle \_ inner}^{4}}$$

is comprised between 7.00×10¹¹ m⁻³ and 1.20×10¹² m⁻³.

In accordance with an embodiment, the outer diameter of the sidewall is for the 29G cannula and the ratio.

$$\frac{{L}_{hydrodynamic theoretical needle}}{{D}_{needle \_ inner}^{4}}$$

is comprised between 2.10×10¹² m⁻³ and 3.50×10¹² m⁻³.

In accordance with an embodiment, the outer diameter of the sidewall is for the 30G

$$\frac{{L}_{hydrodynamic theoretical needle}}{{D}_{needle \_ inner}^{4}}$$

cannula and the ratio is comprised between 4.10×10¹² m⁻³ and 7.00×10¹² m⁻³.

In accordance with an embodiment, the outer diameter of the sidewall is for the 27G cannula and the ratio

$$\frac{{L}_{hydrodynamic theoretical needle}}{{D}_{needle \_ inner}^{4}}$$

is comprised between 1.00×10¹² m⁻³ and 1.60×10¹² m⁻³.

In accordance with an embodiment, the outer diameter of the sidewall is for the 29G cannula and the ratio

$$\frac{{L}_{hydrodynamic theoretical needle}}{{D}_{needle \_ inner}^{4}}$$

is comprised between 3.00×10¹² m⁻³ and 4.70×10¹² m⁻³.

In accordance with an embodiment, the outer diameter of the sidewall is for the 30G cannula and the ratio.

$$\frac{{L}_{hydrodynamic theoretical needle}}{{D}_{needle \_ inner}^{4}}$$

is comprised between 5.70×10¹² m⁻³ and 9.40×10¹² m⁻³.

In accordance with an embodiment, the outer diameter of the sidewall is substantially constant between the proximal end and the distal end.

Also provided herein is a medical injection assembly including a syringe having a sidewall and a distal end, and a cannula connected to the distal end. The cannula further includes a sidewall defining a lumen and having a proximal end and a distal end and a beveled tip defining an opening to the lumen, wherein a flow factor K₁ of a fluid through the lumen of the cannula is approximated by the equation

$$\frac{8 π {D}_{barrel}^{4} {L}_{hydrodynamic theoretical needle}}{{D}_{needle \_ inner}^{4}} ,$$

wherein D_{barrel} is an inner diameter of a sidewall of the syringe, L_{hydrodynamic} is a hydrodynamic length of the cannula, and D_{hydrodynamic} is an empirically derived inner diameter of the sidewall of the cannula, and wherein K₁ is comprised between 7,000 and 31.548×10⁶ meters.

In accordance with an embodiment, the inside volume of the syringe is comprised between 1 mL and 2.25 mL and K₁ is comprised between 35,000 and 1.203×10⁶ meters.

In accordance with an embodiment, the inside volume of the syringe is comprised between 1 mL and 2.25 mL and K₁ is comprised between 29,000 and 1.030×10⁶ meters.

In accordance with an embodiment, the inside volume of the syringe is comprised between 1 mL and 2.25 mL and K₁ is comprised between 41,000 and 1.022×10⁶ meters.

In accordance with an embodiment, the sidewall has a transition region wherein the inner diameter transitions to a break at which an inner diameter of the sidewall is approximately equal to the inner diameter of the cannula.

In accordance with an embodiment, the hydrodynamic length of the cannula extends from the break of the syringe to a proximal end of the opening of the beveled tip.

In accordance with an embodiment, the outer diameter of the sidewall corresponds to a cannula standard for a 27G, 29G or 30G cannula.

In accordance with an embodiment, the outer diameter of the sidewall of the cannula is substantially constant between the proximal end and the distal end.

In accordance with an embodiment, the syringe further includes a stopper slidably disposed within the sidewall of the syringe.

In accordance with an embodiment, the inside volume of the syringe is comprised between 3 mL and 5 mL and K₁ is comprised between 352,000 and 9.948×10⁶ meters.

In accordance with an embodiment, K₁ is comprised between 9,000 and 27.583×10⁶ meters.

In accordance with an embodiment, K₁ is comprised between 7,000 and 23.618×10⁶ meters.

In accordance with an embodiment, K₁ is comprised between 11,000 and 31.548×10⁶ meters.

In accordance with an embodiment, the syringe contains a needle having a length of 12.7 mm, a gauge of 27, a minimum internal diameter of 0.277 mm, and 5 bevels on its distal surface.

In accordance with an embodiment, this invention provides a method of treating a patient having one or more of: allergic asthma; food allergy; chronic rhino sinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering X mL of a 150 mg/mL omalizumab formulation to the patient, wherein X is 1 or 0.5 and wherein the X mL of the 150 mg/mL omalizumab formulation is administered subcutaneously by a syringe, the syringe comprising:
a reservoir filled with the X mL of the 150 mg/mL omalizumab formulation, wherein the X mL of the 150 mg/mL omalizumab formulation has 6000 or fewer particles of diameter >10 μm and/or 600 or fewer particles of diameter >25 μm; and the syringe further comprising:
a stopper, and
a needle having a length of 12.7 mm, a gauge of 27, a minimum internal diameter of 0.277 mm, and 5 bevels on its distal surface; and
the syringe being configured to expel the omalizumab formulation contained in the reservoir through the needle when a force is applied to the stopper, wherein one of the following options is fulfilled when a constant force of 10N is applied to the stopper:
a) when X is 1, the syringe is configured to expel the omalizumab formulation contained in the reservoir through the needle in less than 5 seconds; or
b) when X is 0.5, the syringe is configured to expel the omalizumab formulation contained in the reservoir through the needle in less than 4 seconds.

In accordance with an embodiment, this invention provides a syringe comprising: a reservoir filled with X mL of a 150 mg/mL omalizumab formulation, wherein X is 2, 1 or 0.5, wherein the X mL of the 150 mg/mL omalizumab formulation has 6000 or fewer particles of diameter >10 μm and/or 600 or fewer particles of diameter >25 μm; and the syringe further comprising:
a stopper, and
a needle having a length of 12.7 mm, a gauge of 27, a minimum internal diameter of 0.277 mm, and 5 bevels on its distal surface, the syringe being configured to expel the X mL of the omalizumab formulation contained in the reservoir through the needle when a force is applied to the stopper, wherein one of the following options is fulfilled when a constant force of 10N is applied to the stopper:
a) when X is 2, the syringe is configured to expel the omalizumab formulation contained in the reservoir through the needle in less than 16 seconds;
b) when X is 1, the syringe is configured to expel the omalizumab formulation contained in the reservoir through the needle in less than 5 seconds; or
c) when X is 0.5, the syringe is configured to expel the omalizumab formulation contained in the reservoir through the needle in less than 4 seconds.

Further details and advantages of the various examples described in detail herein will become clear upon reviewing the following detailed description of the various examples in conjunction with the accompanying drawing figures.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a schematic of a cannula attached to a distal end of a syringe barrel;

FIG. 2 is a top view of the cannula of FIG. 1;

FIG. 3 is a graph illustrating acceptable length-to-diameter ratios of 27G, 29G and 30G cannulas of 8 mm exposed length, according to aspects or embodiments of the present disclosure;

FIG. 4 is a graph illustrating acceptable flow factors K₁ of 1 mLL and 2.25 mLL syringes having a 27G, 29G or 30G cannula of 8 mm exposed length, according to aspects of the disclosure;

FIG. 5 is a front view of a medical injection assembly according to one aspect or embodiment to the present disclosure;

FIG. 6 is a cross-sectional view of the medical injection assembly of FIG. 5;

FIG. 7 is a front view of a drug delivery device; and

FIG. 8 is a front view of a drug delivery device.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

All numbers used in the specification and claims are to be understood as being modified in all instances by the term “about”. The terms “approximately”, “about”, and “substantially” mean a range of plus or minus ten percent of the stated value. Further, the term “substantially equal” and like terms mean that the compared values or dimensions are within a range of plus or minus ten percent of one another.

The terms “first”, “second”, and the like are not intended to refer to any particular order or chronology, but refer to different conditions, properties, or elements.

As used herein with reference to an injection apparatus such as a syringe, the term “proximal” refers to an end of the apparatus farthest from the outlet, or to a direction toward the end of the apparatus farthest from the outlet. As used herein with reference to an injection apparatus such as a syringe, the term “distal” refers to an end of the device or apparatus closest to the outlet, or to a direction toward the end of the apparatus closest to the outlet.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

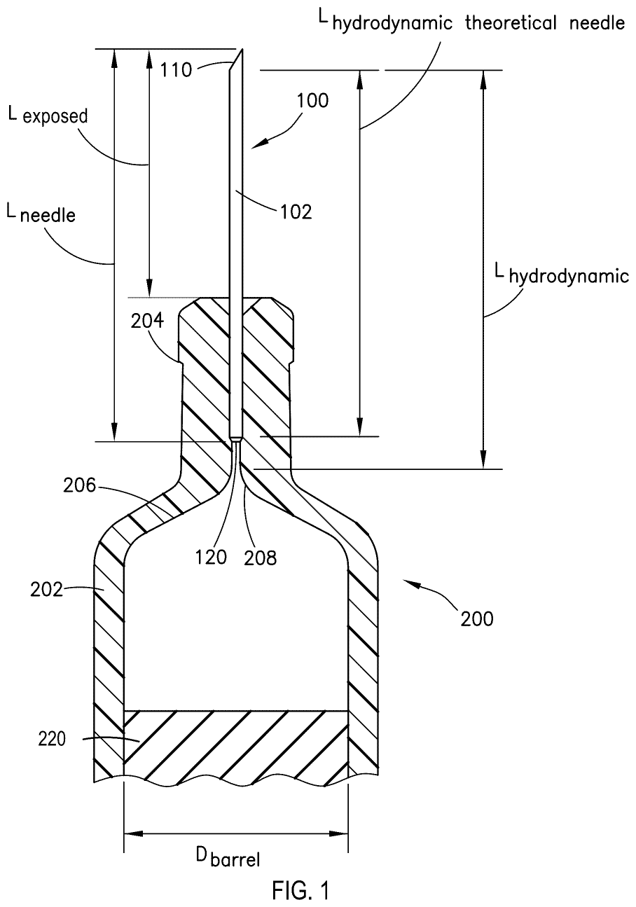

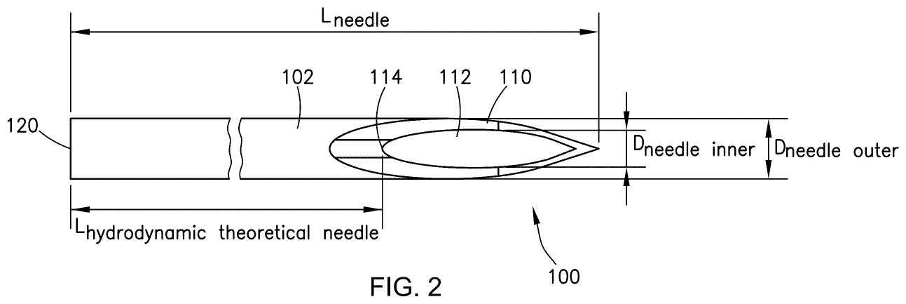

Referring now to FIGS. 1 and 2, a cannula 100 in accordance with the present disclosure includes a sidewall 102 extending between a distal end 110 and a proximal end 120. The sidewall 102 has an outer diameter D_{needle_outer} and an inner diameter D_{needle_inner}, with a difference between the outer diameter D_{needle_outer} and the inner diameter D_{needle_inner} defining a wall thickness. The inner diameter D_{needle_inner} of the sidewall 102 defines a lumen whose cross-section is substantially circular and through which a medicament, blood, or other fluid can flow. The outer diameter D_{needle_outer} of the sidewall 102 may be equivalent to industry-standard nominal sizes for a selected cannula gauge (G), with the gauge size defining the limits for the outer diameter. As non-limiting examples, a 27G cannula will have an outer diameter of between 0.400 and 0.420 mm, a 29G cannula will have an outer diameter of between 0.324 and 0.351 mm, and a 30G cannula will have an outer diameter of between 0.298 and 0.320 mm. The outer diameter D_{needle_outer} of the sidewall 102 may be substantially constant between the proximal end 120 and the distal end 110. The distal end 110 may include a beveled tip configured for piercing a patient's skin to create a vascular access site. The beveled tip defines an opening 112 into the lumen. The opening has a proximal end 114. The proximal end 120 of the cannula 100 is configured for connection to a distal end 204 of a syringe 200. The overall length L_{needle} of the cannula 100 extends from the proximal-most end of the sidewall 102 to the distal-most end of the sidewall 102, including the length of the beveled tip of the distal end 110.

As shown in FIG. 1, the syringe 200 (which may be formed of plastic or glass, typically glass) includes a sidewall 202 having an inner diameter D_{barrel}. The inner diameter D_{barrel} depends on the volume of the barrel and the associated volume of drug to be injected. For instance, the barrel may be a 1 mLL (1 mm long) or 2.25 mL barrel, with the 1 mLL barrel having an inner diameter D_{barrel} of 6.35 mm+/−0.1 mm and the 2.25 mL barrel having an inner diameter D_{barrel} of 8.65 mm+/−0.2 mm. The sidewall 202 tapers toward the distal end 204 of the syringe 200 where the cannula 100 is connected (e.g., glued). In particular, the sidewall 202 tapers in a transition region 206 from the inner diameter D_{barrel} to a smaller diameter slightly lower than the outer diameter D_{needle_outer} of the cannula 100. The transition region 206 ends at a break 208, at which point the inner diameter of the sidewall 202 is approximately equal to the inner diameter D_{needle_inner} of the cannula 100. An elastomeric stopper 220 is slidably disposed within the sidewall 202 and can be advanced distally to expel fluid from the syringe 200 through the cannula 100. The stopper 220 may be affixed to the end of a plunger (not shown) that can be depressed by a clinician's finger(s).

Different lengths of the cannula 100 are significant to measuring and characterizing the flow rate of a fluid through the lumen of the cannula 100. First, the aforementioned overall length L_{needle} extends from the proximal-most end of the sidewall 102 to the distal-most end of the sidewall 102. Second, the theoretical hydrodynamic length L_{hydrodynamic theoretical needle} extends from the proximal-most end of the sidewall 102 to the proximal end 114 of the opening 112 of the beveled tip. Third, the actual hydrodynamic length L_{hydrodynamic} extends from the break 208 of the syringe 200 (where the diameter of the barrel is the smallest) to the proximal end 114 of the opening 112 of the beveled tip. Fourth, the exposed needle length L_{exposed} is a length of the cannula 100 that extends beyond a distal-most end of the syringe 200. In some aspects or embodiments, the exposed needle length L_{exposed} is 6 mm, 8 mm, or 10 mm. A length of the portion of the cannula 100 positioned within the syringe 200 may be the same regardless of the exposed needle length L_{exposed}.

Injection force required to inject a fluid from the syringe 200 through the cannula 100 is a complex calculation involving many factors. However, this calculation can be simplified for many relevant applications so long as stopper speed within the syringe is not abnormally high, and so long as dynamic viscosity of the fluid injected in not abnormally low. In particular, the calculation of an injection force F can be simplified as shown below in Equation 1:

$$F = \frac{8 π {D}_{barrel}^{4} {L}_{hydrodynamic}}{{D}_{hydrodynamic}^{4}} \times η \times {v}_{stopper} + R , \qquad Equation 1$$

where F is injection force, η is viscosity of the fluid injected, vₛₜₒₚₚₑᵣ is the speed of the stopper in the syringe 200, and R is force due to friction of the stopper.

The terms in Equation 1 that are derived from the physical dimensions of the cannula 100 and barrel 200 can be gathered into a flow factor K₁, as shown below in Equation 2:

$${K}_{1} = \frac{8 π {D}_{barrel}^{4} {L}_{hydrodynamic}}{{D}_{hydrodynamic}^{4}} . \qquad Equation 2$$

In other embodiments, where a theoretical hydrodynamic needle length L_{hydrodynamic theoretical needle} and inner diameter D_{needle_inner} define the dimension of the cannula 100, the flow factor K₁, may be as shown below in Equation 3:

$${K}_{1} = \frac{8 π {D}_{barrel}^{4} {L}_{hydrodynamic theoretical needle}}{{D}_{needle \_ inner}^{4}} . \qquad Equation 3$$

The flow factor K₁ is presented in units of distance and, for most cannulas used in medical devices, is typically in a range of 10,000 meters and 30,000,000 meters. The flow factor K₁ is related to injection time, with a relatively lower flow factor K₁ correlating to a faster injection time and a relatively higher flow factor K₁ correlating to a longer injection time for the same volume of fluid injected. In order to optimize the injection of a drug, it is desirable to have a lower factor K₁. Indeed, when the factor K₁ is lowered, the injection force required to inject the drug contained into the syringe is lowered too. In lowering the required injection force, the injection is easier to perform and/or a more viscous drug may be injected into a patient's body, such as a subcutaneous injection, without considerably increasing the required injection force. While maximizing the flow factor K₁ can be readily accomplished by increasing the outer diameter, it is desirable to minimize the outer diameter of the cannula 100 for patient comfort and perception. Accordingly, the cannula 100 according to aspects and embodiments of the present application provides a balance between minimizing the outer diameter while maximizing the inner diameter.

In addition to the flow factor K₁, the cannula 100 according to the present disclosure can be characterized by what may generally be termed a “length-to-diameter ratio” of the cannula—with the length-to-diameter ratio defined herein as a ratio of the theoretical hydrodynamic needle length to the fourth power of the inner diameter (L_{hydrodynamic} theoretical needle/D_{needle_inner}⁴) or as a ratio of the actual hydrodynamic needle length to the fourth power of the hydrodynamic diameter (L_{hydrodynamic}/D_{hydrodynamic}⁴).

The inner diameter D_{needle_inner} of the cannula 100 is typically ascertained from a dimensional drawing of the cannula 100, and, due to manufacturing limitations, is not necessarily reflective of a measurement from the actual cannula 100. In order to measure small diameter bores such as cannula lumens, gauge pins are typically used. However, gauge pins have their own limitations, notably in that gauge pins are incapable of reliably measuring a tapered bore and other inconsistencies in the bore introduced by manufacturing. Thus, instead of relying on physically measuring the inner diameter D_{needle_inner} of the cannula 100, the hydrodynamic diameter D_{hydrodynamic} can be experimentally derived by pushing fluid through the cannula at a known or deduced flow rate. In some embodiments, the flow rate may be deduced by measuring the mass of fluid during a given time period and by knowing the density of the fluid. Based on this flow rate, the ratio of the actual hydrodynamic needle length to the fourth power of the hydrodynamic diameter (L_{hydrodynamic}/D_{hydrodynamic}⁴) can be deduced via the fluid viscosity and test pressure. If the actual hydrodynamic length L_{hydrodynamic} of the needle is known, the hydrodynamic diameter D_{hydrodynamic} may then be deduced.

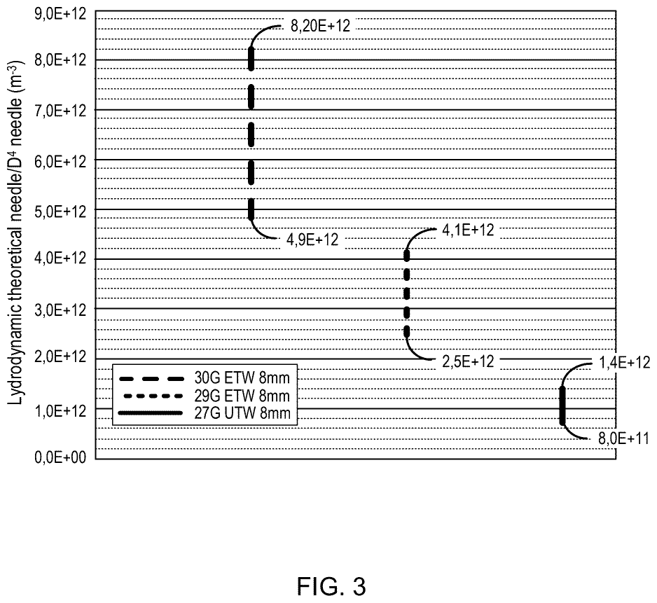

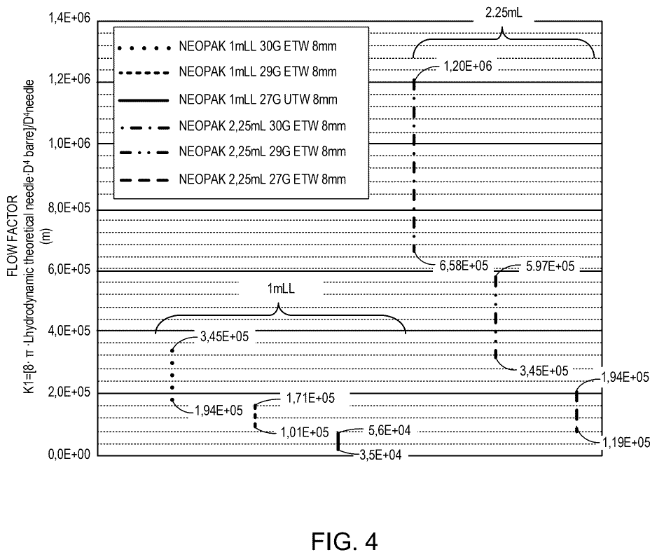

Referring now to FIGS. 3 and 4, graphs are provided illustrating the length-to-diameter ratio (i.e., the ratio of the theoretical hydrodynamic needle length to the fourth power of the inner diameter, L_{hydrodynamic theoretical needle}/D_{needle_inner}⁴) and the flow factor K₁, respectively, for various syringes that include a cannula having an 8 mm exposed needle length L_{exposed}, according to aspects of the disclosure. The selection of the length-to-diameter ratio of the cannula 100, according to aspects or embodiments of the present application, is a compromise between the length-to-diameter ratio and the outer diameter of the cannula to ensure the outer diameter meets patient's expectations with respect to size and comfort. Accordingly, while the length-to-diameter ratio for a 30G cannula may be higher than a 27G cannula, the outer diameter of the 30G cannula is smaller and it is desirable to find a compromise between the outer diameter of the cannula and the length-to-diameter ratio.

Referring first to FIG. 3, an acceptable range of the length-to-diameter ratio (L_{hydrodynamic} theoretical needle/D_{needle_inner}⁴) is provided for each of a 27G, 29G and 30G cannula (for an exposed needle length L_{exposed} of 8 mm). As shown therein, the length-to-diameter ratio of the 27G cannula is between 8.00×10¹¹ m⁻³ and 1.40×10¹² m⁻³, the length-to-diameter ratio of the 29G cannula is between 2.50×10¹² m⁻³ and 4.10×10¹² m⁻³, and the length-to-diameter ratio of the 30G cannula is between 4.90×10¹² m⁻³ and 8.20×10¹² m⁻³. An acceptable range of the length-to-diameter ratio across all of the or 27G, 29G and 30G cannulas is thus between 8.00×10¹¹ and 8.20×10¹² m⁻³, according to aspects of the present disclosure.

Referring next to FIG. 4, an acceptable range of a flow factor K₁ is provided for 1 mLL syringes having a 27G, 29G or 30G cannula and for 2.25 mL syringes having a 27G, 29G or 30G cannula (for an exposed needle length L_{exposed} of 8 mm). As shown therein, the flow factor K₁ of the 1 mLL syringes is between 35,000 m and 56,000 m for the 27G cannula, 101,000 m and 171,000 m for the 29G cannula, and 194,000 m and 345,000 m for the 30G cannula, while the flow factor K₁ of the 2.25 mL syringes is between 119,000 m and 194,000 m for the 27G cannula, 345,000 m and 596,000 m for the 29G cannula, 658,000 m and 1.20×10⁶ m for the 30G cannula. An acceptable range of the flow factor K₁ across the 1 mLL and 2.25 mL syringes and all of the 27G, 29G and 30G cannulas is thus between 35,000 and 1.20×10⁶ meters.

According to a further embodiment, acceptable ranges of the flow factor K₁ for 0.5 mL syringes, 1 mLL syringes, 2.25 mL syringes, 3 mL syringes, 5 mL syringes, and 10 mL syringes with a 27G 8 mm exposed needle length L_{exposed}, 29G 8 mm exposed needle length L_{exposed}, or 30G 8 mm exposed needle length L_{exposed} cannula is shown in Table 1 below.

TABLE 1
Summary of Flow Factor K₁ Values (8 mm exposed needle
length L_{exposed} cannula) - values in meters.

| | | K1 min | K1 max | K1 min | K1 max | K1 min | K1 max |
|---|---|---|---|---|---|---|---|
| | | 27G UTW | 27G UTW | 29G ETW | 29G ETW | 30G ETW | 30G ETW |
| | | 8 mm | 8 mm | 8 mm | 8 mm | 8 mm | 8 mm |
| 0.5 | mL | 9,000 | 17,000 | 27,000 | 52,000 | 52,000 | 105,000 |
| 1 | mlL | 35,000 | 56,000 | 101,000 | 171,000 | 194,000 | 345,000 |
| 2.25 | mL | 119,000 | 194,000 | 345,000 | 597,000 | 658,000 | 1,203,000 |
| 3 | mL | 426,000 | 675,000 | 1,231,000 | 2,076,000 | 2,347,000 | 4,183,000 |
| 5 | mL | 896,000 | 1,403,000 | 2,590,000 | 4,317,000 | 4,937,000 | 8,697,000 |
| 10 | mL | 2,844,000 | 4,449,000 | 8,334,000 | 13,689,000 | 15,884,000 | 27,583,000 |

According to a further embodiment, acceptable ranges of the flow factor K₁ for 0.5 mL syringes, 1 mLL syringes, 2.25 mL syringes, 3 mL syringes, 5 mL syringes, and 10 ml syringe with a 27G 6 mm exposed needle length L_{exposed}, 29G 6 mm exposed needle length L_{exposed}, or 30G 6 mm exposed needle length L_{exposed} cannula is shown in Table 2 below.

TABLE 2
Summary of Flow Factor K₁ Values (6 mm exposed needle
length L_{exposed} cannula) - values in meters.

| | | K1 min | K1 max | K1 min | K1 max | K1 min | K1 max |
|---|---|---|---|---|---|---|---|
| | | 27G UTW | 27G UTW | 29G ETW | 29G ETW | 30G ETW | 30G ETW |
| | | 6 mm | 6 mm | 6 mm | 6 mm | 6 mm | 6 mm |
| 0.5 | mL | 7,000 | 15,000 | 23,000 | 45,000 | 44,000 | 90,000 |
| 1 | mlL | 29,000 | 48,000 | 84,000 | 146,000 | 162,000 | 295,000 |
| 2.25 | mL | 98,000 | 165,000 | 288,000 | 510,000 | 550,000 | 1,030,000 |
| 3 | mL | 352,000 | 574,000 | 1,027,000 | 1,774,000 | 1,964,000 | 3,582,000 |
| 5 | mL | 741,000 | 1,193,000 | 2,160,000 | 3,688,000 | 6,950,000 | 7,447,000 |
| 10 | mL | 2,386,000 | 3,784,000 | 6,950,000 | 11,695,000 | 13,287,000 | 23,618,000 |

According to a further embodiment, acceptable ranges of the flow factor K₁ for 0.5 mL syringes 1 mLL syringes, 2.25 mL syringes, 3 mL syringes, 5 mL syringes, and 10 ml syringe with a 27G 10 mm exposed needle length L_{exposed}, 29G 10 mm exposed needle length L_{exposed}, or 30G 10 mm exposed needle length L_{exposed} cannula is shown in Table 3 below.

TABLE 3
Summary of Flow Factor K₁ Values (10 mm exposed needle
length L_{exposed} cannula) - values in meters.

| | | K1 min | K1 max | K1 min | K1 max | K1 min | K1 max |
|---|---|---|---|---|---|---|---|
| | | 27G UTW | 27G UTW | 29G ETW | 29G ETW | 30G ETW | 30G ETW |
| | | 10 mm | 10 mm | 10 mm | 10 mm | 10 mm | 10 mm |
| 0.5 | mL | 11,000 | 20,000 | 32,000 | 60,000 | 61,000 | 120,000 |
| 1 | mlL | 41,000 | 64,000 | 118,000 | 196,000 | 225,000 | 394,000 |
| 2.25 | mL | 140,000 | 223,000 | 402,000 | 684,000 | 765,000 | 1,022,000 |
| 3 | mL | 499,000 | 776,000 | 1,436,000 | 2,379,000 | 2,731,000 | 4,784,000 |
| 5 | mL | 1,051,000 | 1,613,000 | 3,021,000 | 4,945,000 | 5,745,000 | 9,948,000 |
| 10 | mL | 3,381,000 | 5,114,000 | 9,717,000 | 15,683,000 | 18,480,000 | 31,548,000 |

According to a further embodiment, acceptable ranges of the length-to-diameter ratio (L_{hydrodynamic} theoretical needle/D_{needle_inner}⁴) for 8 mm, 10 mm, and 6 mm exposed needle length L_{exposed} 27G, 29G and 30G cannulas is shown in Table 4 below.

TABLE 4
Summary of Length-to-Diameter Ratio (8 mm, 10 mm, and 6 mm
exposed needle length L_{exposed} cannulas) - values in m⁻³.

| | 8 mm | | 10 mm | |
|---|---|---|---|---|
| | L_{hydro}/D_{needle}_{_{—}}ᵢₙₙₑᵣ_{⁴} | L_{hydro}/D_{needle}_{_{—}}ᵢₙₙₑᵣ_{⁴} | L_{hydro}/D_{needle}_{_{—}}ᵢₙₙₑᵣ_{⁴} | L_{hydro}/D_{needle}_{_{—}}ᵢₙₙₑᵣ_{⁴} |
| | min | max | min | max |
| 27G | 8.00 × 10¹¹ | 1.40 × 10¹² | 1.00 × 10¹² | 1.60 × 10¹² |
| UTW | | | | |
| 29 G | 2.50 × 10¹² | 4.10 × 10¹² | 3.00 × 10¹² | 4.70 × 10¹² |
| ETW | | | | |
| 30G | 4.90 × 10¹² | 8.20 × 10¹² | 5.70 × 10¹² | 9.40 × 10¹² |
| ETW | | | | |

| | 6 mm | |
|---|---|---|
| | L_{hydro}/D_{needle}_{_{—}}ᵢₙₙₑᵣ_{⁴} | L_{hydro}/D_{needle}_{_{—}}ᵢₙₙₑᵣ_{⁴} |
| | min | max |
| 27G | 7.00 × 10¹¹ | 1.20 × 10¹² |
| UTW | | |
| 29 G | 2.10 × 10¹² | 3.50 × 10¹² |
| ETW | | |
| 30G | 4.10 × 10¹² | 7.00 × 10¹² |
| ETW | | |

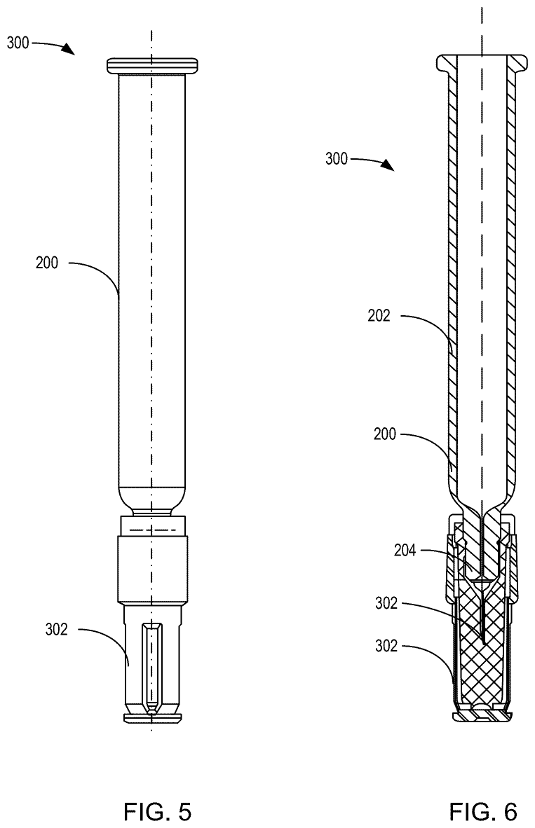

Referring to FIGS. 5 and 6, a medical injection assembly 300 includes the syringe 200 and the cannula 100 discussed above and shown in FIGS. 1 and 2. The medical injection assembly 300 also includes a rigid needle shield (RNS) 302 that receives at least the distal end 110 of the cannula 100 and is engaged with the distal end 204 of the syringe 200. The RNS 302 is configured to be removed from the syringe 200 prior to use of the syringe 200. Although not shown in FIG. 6, the syringe 200 includes the stopper 220. In some aspects or embodiments, the syringe 200 is prefilled with a fluid or medication with the stopper 220 sealing and closing the open end of the syringe 200.

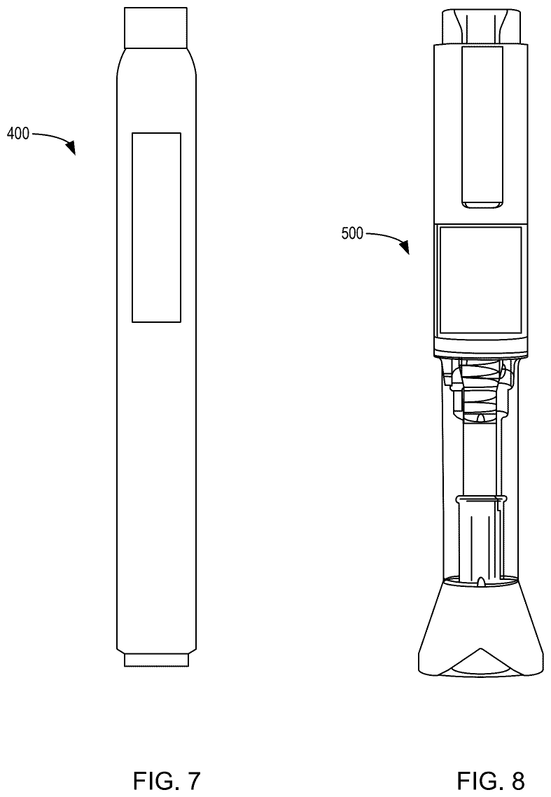

Referring to FIGS. 7 and 8, the syringe 200, the cannula 100, and/or the medical injection assembly 300 are configured to be utilized with a drug delivery device, such as an auto-injector 400. In one aspect or embodiment, the drug delivery device is an auto-injector 500 (FIG. 8), such as the BD Physioject™ disposable autoinjector commercially available from Becton, Dickinson and Company.

In one embodiment, the needle has a gauge of 25, the needle has one of: a) a minimum internal diameter of 0.292 mm, or b) a minimum internal diameter of 0.232 mm.

In one embodiment, the needle has a gauge of 26, the needle has one of: a) a minimum internal diameter of 0.292 mm; or b) a minimum internal diameter of 0.232 mm.

In one embodiment, the needle has a gauge of 27, the needle has one of: a) a minimum internal diameter of 0.277 mm; b) a minimum internal diameter of 0.191 mm; c) a minimum internal diameter of 0.184 mm; or d) a minimum internal diameter of 0.241 mm.

In one embodiment, the needle has a gauge of 28, the needle has one of: a) a minimum internal diameter of 0.133 mm; or b) a minimum internal diameter of 0.190 mm.

In one embodiment, the needle has a gauge of 29, the needle has one of: a) a minimum internal diameter of 0.265 mm; b) a minimum internal diameter of 0.240 mm; c) a minimum internal diameter of 0.190 mm; or d) a minimum internal diameter of 0.133 mm.

In some embodiments of the invention, the needle has 3 bevels on its distal surface or 5 bevels on its distal surface.

In some embodiments of the invention, the syringe may include at least 0.4 mg/ml polysorbate 20 after filling with 150 mg/mL omalizumab solution stored at 5° C.±1° C.

It is another embodiment of the invention wherein the syringe has an internal coating of 0.4±0.2 mg of polydimethylsiloxane.

It is another embodiment of the invention wherein the syringe has a latex-free needle shield, or a round flange at a proximal end of the reservoir. In another embodiment, the round flange has a maximum diameter of 11±0.25 mm or 14.7±0.25 mm.

In some embodiments of the invention, the syringe is provided in a needle shield device comprising a needle sleeve and a plunger; and wherein a manual force can be applied to the plunger to expel the 150 mg/mL omalizumab formulation and unlatch the needle sleeve so that the needle sleeve can cover the needle after injection.

In some embodiments of the invention, the syringe has a length of 54.0±0.5 mm and an internal diameter of 6.35 mm±0.05 mm.

In some embodiments of the invention, the syringe has a length of 54.0±0.5 mm and an internal diameter of 8.65 mm±0.05 mm.

In some embodiments of the invention, the syringe is provided in a needle shield device comprising a needle sleeve and a plunger; and wherein a manual force can be applied to the plunger to expel the 150 mg/mL omalizumab formulation and unlatch needle sleeve so that the needle sleeve can cover the needle after injection.

In some embodiments of the invention, the syringe includes a stopper. In some embodiments of the invention, the stopper has a fluoro-resin on the product contacting side or an ethylene tetrafluoroethylene (ETFE) barrier film lamination. In some embodiments of the invention, the stopper has a B2-40 UV cured lubrication coating or is lubricated with 1000 cSt silicone oil.

In some embodiments, when the stopper is not inserted into the reservoir, it has one of: a) a maximum external diameter of 6.67 mm to 7.10 mm and/or a length of 7.85±0.4 mm; or b) a maximum external diameter of 6.70±0.15 mm and/or a length of 7.85±0.4 mm.

In some embodiments of the invention, the stopper has at least one circumferential rib of outer diameter 6.60±0.15 mm. In some embodiments of the invention, the stopper has at least one circumferential rib of outer diameter 9.00±0.15 mm.

In some embodiments of the invention, a break loose force configured to move the stopper from an initial stationary point to a velocity of 190 mm/min is one or more of: a) about 2N to about 3.5N at 25° C.±1° C. after filling; b) about 2N to about 4.5N after storage at 25° C.±1° C. for 1.5 months after filling; c) about 2.5N to about 5N after storage at 25° C.±1° C. for 2.5 months after filling; or d) about 2.5N to about 5.5N after storage at 25° C.±1° C. for 6 months after filling.

In another embodiment of the invention, the stopper has a B2-40 UV cured lubrication coating or is lubricated with 1000 cSt silicone oil.

In another embodiment of the invention, the stopper has a maximum external diameter 9.05±0.15 mm and/or length 7.70±0.4 mm when not inserted into the reservoir.

In one embodiment of the invention, the syringe is configured to expel the X mL of the 150 mg/mL omalizumab formulation contained in the reservoir through the needle when a constant force of 10N is applied to the stopper wherein a) when X is 1, the syringe is configured to expel the omalizumab formulation contained in the reservoir through the needle in less than 5 seconds, or when X is 0.5, the syringe is configured to expel the omalizumab formulation contained in the reservoir through the needle in less than 4 seconds.

In another embodiment of the invention, the syringe is configured to expel the X mL of the 150 mg/mL omalizumab formulation contained in the reservoir through the needle, wherein X is 1, and wherein the syringe is configured to expel the X mL of the 150 mg/ml omalizumab formulation through the needle in one or more of: a) less than 5 seconds when a constant force of 10N is applied to the stopper; b) less than 3 seconds when a constant force of 17N is applied to the stopper; or c) less than 2 seconds when a constant force of 30N is applied to the stopper.

In another embodiment of the invention, the syringe is configured to expel the X mL of the 150 mg/mL omalizumab formulation contained in the reservoir through the needle, wherein X is 1, and wherein the syringe is configured to expel the X mL of the 150 mg/ml omalizumab formulation through the needle in one or more of, a) about 4 seconds to less than 5 seconds when a constant force of 10N is applied to the stopper; b) about 2 seconds to less than 3.5 seconds when a constant force of 17N is applied to the stopper; or c) about 1 second to less than 2 seconds when a constant force of 30N is applied to the stopper.

In another embodiment of the invention, the syringe is configured to expel the X mL of the 150 mg/mL omalizumab formulation contained in the reservoir through the needle, wherein X is 0.5, wherein the syringe is configured to expel the X mL of the 150 mg/ml omalizumab formulation through the needle in one or more of: a) less than 2 seconds when a constant force of 17N is applied to the stopper; or b) less than 1 second when a constant force of 30N is applied to the stopper.

In another embodiment of the invention, the syringe is configured to expel the X mL of the 150 mg/mL omalizumab formulation contained in the reservoir through the needle, wherein X is 0.5, wherein the syringe is configured to expel the X mL of the 150 mg/ml omalizumab formulation through the needle in one or more of: a) about 2 seconds to less than 4 seconds when a constant force of 10N is applied to the stopper; b) about 1 second to less than 2 seconds when a constant force of 17N is applied to the stopper; or c) about 0.5 seconds to less than 1 second when a constant force of 30N is applied to the stopper.

In another embodiment of the invention, the syringe is configured to expel the omalizumab formulation contained in the reservoir through the needle in one or more of: a) less than 8.5 seconds when a constant force of 17N is applied to the stopper; or b) less than 5 seconds when a constant force of 30N is applied to the stopper.

In another embodiment of the invention, the syringe is configured to expel X mL of the omalizumab formulation contained in the reservoir through the needle in one or more of: a) about 16 seconds to about 14 seconds when a constant force of 10N is applied to the stopper; b) about 8.5 seconds to about 6 seconds when a constant force of 17N is applied to the stopper; or c) about 5 seconds to about 4 seconds when a constant force of 30N is applied to the stopper.

In one embodiment, the syringe 200 may be prefilled with a composition for treatment of one or more of the following indications: Rheumatoid Arthritis, Plaque Psoriasis (Psoriasis Vulgaris), Psoriatic Arthritis, Ulcerative Colitis, Ankylosing Spondylitis (Bekhterev's Disease), Atopic Dermatitis (Atopic Eczema), Allergic Rhinitis, Asthma, Allergic Rhino-Conjunctivitis, Allergic Asthma, Polyarticular Juvenile Idiopathic Arthritis (PJIA), Psoriasis, Axial Spondylarthrites, Hidradenitis Suppurativa, Grass Pollen Allergy, Food Allergy, Chronic Urticaria, Hives, Diarrhea, Autoimmune Disease, Obstructive Pulmonary Disease (COPD), Endometriosis, Ovarian Hyperstimulation Syndrome, Nasal Polyps (Nasal Polyposis), Uveitis, House Dust Mite Allergy, and/or Seasonal Allergic Rhinitis.

In one embodiment, the syringe 200 may be prefilled with a composition including one or more of the following: allergy medications, antihistamines, anti-inflammatoi bronchodilators, cold cures, corticosteroids, cough suppressants, decongestants, expectorant, hormones, immunosuppressives, sleep aids, tranquilizers, and vitamins.

In one embodiment, the syringe 200 may include one or more biologics, such as omalizumab, anti-immunoglobin E Antibodies or other recombinant DNA-derived monoclonal antibodies, such as omalizumab-igec, dupilumab, mepolizumab, benralizumab, and tezepelumab-ekko, and/or other equivalents. In one embodiment, the syringe 200 may include one or more of adalimumab-afzb, tocilizumab, tralokinumab-ldrm, adalimumab-atto, bimekizumab-bkzx, secukinumab, adalimumab-adbm, etanercept, vedolizumab, etanercept-szzs, etanercept-ykro, benralizumab, adalimumab-bwwd, adalimumab-fkjp, adalimumab, adalimumab-adaz, adalimumab-aacf, tildrakizumab, sarilumab, anakinra, mirikizumab-mrkz, ustekinumab-ttwe, brodalumab, adalimumab-ryvk, golimumab, risankizumab-rzaa, ustekinumab, ixekizumab, tezepelumab-ekko, guselkumab, ustekinumab-auub, omalizumab, adalimumab-aaty, adalimumab-aqvh, infliximab-dyyb, certolizumab, ustekinumab-aekn, Animal Allergens, Standardized Cat Hair, infliximab-abda, infliximab-axxq, spesolimab-sbzo, and/or adalimumab-aaty, mirikizumab-mrkz.

In one embodiment, the syringe 200 may include monoclonal antibodies. In one embodiment, the syringe 200 may include one or more of erenumab, fremanezumab-vfrm, garadacimab-gxii, and/or immunoglobin E antibodies.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A method of treating a patient having one or more of: allergic asthma; food allergy; chronic rhino sinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering X mL of a 150 mg/mL omalizumab formulation to the patient, wherein X is 1 or 0.5 and wherein the X mL of the 150 mg/mL omalizumab formulation is administered subcutaneously by a syringe, the syringe comprising:
a reservoir filled with the X mL of the 150 mg/mL omalizumab formulation, wherein the X mL of the 150 mg/mL omalizumab formulation has 6000 or fewer particles of diameter >10 μm and/or 600 or fewer particles of diameter >25 μm; and the syringe further comprising:
a stopper, and
a needle having a length of 12.7 mm, a gauge of 27, a minimum internal diameter of 0.277 mm, and 5 bevels on its distal surface; and
the syringe being configured to expel the omalizumab formulation contained in the reservoir through the needle when a force is applied to the stopper, wherein one of the following options is fulfilled when a constant force of 10N is applied to the stopper:
a) when X is 1, the syringe is configured to expel the omalizumab formulation contained in the reservoir through the needle in less than 5 seconds; or
b) when X is 0.5, the syringe is configured to expel the omalizumab formulation contained in the reservoir through the needle in less than 4 seconds.

2. The method of claim 1, wherein X is 1, and wherein the syringe is configured to expel the X mL of the 150 mg/ml omalizumab formulation through the needle in one or more of:
a) less than 5 seconds when a constant force of 10N is applied to the stopper;
b) less than 3 seconds when a constant force of 17N is applied to the stopper; or
c) less than 2 seconds when a constant force of 30N is applied to the stopper.

3. The method of claim 2, wherein the syringe is configured to expel the X mL of the 150 mg/ml omalizumab formulation through the needle in one or more of:
a) about 4 seconds to less than 5 seconds when a constant force of 10N is applied to the stopper;
b) about 2 seconds to less than 3.5 seconds when a constant force of 17N is applied to the stopper; or
c) about 1 second to less than 2 seconds when a constant force of 30N is applied to the stopper.

4. The method of claim 1, wherein X is 0.5, wherein the syringe is configured to expel the X mL of the 150 mg/ml omalizumab formulation through the needle in one or more of:
a) less than 2 seconds when a constant force of 17N is applied to the stopper; or
b) less than 1 second when a constant force of 30N is applied to the stopper.

5. The method of claim 4, wherein the syringe is configured to expel the X mL of the 150 mg/ml omalizumab formulation through the needle in one or more of:
a) about 2 seconds to less than 4 seconds when a constant force of 10N is applied to the stopper;
b) about 1 second to less than 2 seconds when a constant force of 17N is applied to the stopper; or
c) about 0.5 seconds to less than 1 second when a constant force of 30N is applied to the stopper.

6. The method of claim 1, wherein the syringe contains:
a) at least 76% main omalizumab charge variant measured by ion exchange chromatography after being filled with 150 mg/mL omalizumab solution stored at 5° C.±1° C.; or
b) at least 77% main omalizumab charge variant measured by ion exchange chromatography after being filled with 150 mg/mL omalizumab solution and then stored at 5° C.±1° C. for 2.5 months.

7. The method of claim 1, wherein the proportion of the first peak of the syringe measured by hydrophobic interaction chromatography is at least:
a) 62% after filling with 150 mg/mL omalizumab solution stored at 5° C.±1° C.; or
b) 62% after being filled with 150 mg/mL omalizumab solution and then stored at 5° C.±1° C. for 2.5 months.

8. The method of claim 1, wherein the syringe contains at least 0.4 mg/mL polysorbate 20 after filling with 150 mg/mL omalizumab solution stored at 5° C.±1° C.

9. The method of claim 1, wherein the stopper has a fluoro-resin on the product contacting side or an ethylene tetrafluoroethylene (ETFE) barrier film lamination.

10. The method of claim 1, wherein the stopper has a B2-40 UV cured lubrication coating or is lubricated with 1000 cSt silicone oil.

11. The method of claim 1, wherein the syringe has an internal coating of 0.4±0.2 mg of polydimethylsiloxane.

12. The method of claim 1, wherein the apparent viscosity of the 150 mg/ml omalizumab formulation is 12 to 14 mPa·s or, when measured at a shear rate of 200 s⁻¹ is: 24.7±0.5 m·Pas at 5.0±0.2° C., 15.3±0.5 m·Pas at 15.0±0.2° C., 11.4±0.5 mPa·s at 25.0±0.2° C., or 7.2±1.2 m·Pas at 40.0±0.2° C.

13. The method of claim 1, wherein the syringe has a latex-free needle shield.

14. The method of claim 1, wherein the syringe has a round flange at a proximal end of the reservoir.

15. The method of claim 1, wherein the syringe has a length of 54.0±0.5 mm and an internal diameter of 6.35 mm±0.05 mm.

16. The method of claim 1, wherein when the stopper is not inserted into the reservoir, it has one of:
a) a maximum external diameter of 6.67 mm to 7.10 mm and/or a length of 7.85±0.4 mm; or
b) a maximum external diameter of 6.70±0.15 mm and/or a length of 7.85±0.4 mm.

17. The method of claim 1, wherein the stopper has at least one circumferential rib of outer diameter 6.60±0.15 mm.

18. The method of claim 1, wherein a break loose force configured to move the stopper from an initial stationary point to a velocity of 190 mm/min is one or more of:
a) about 2N to about 3.5N at 25° C.±1° C. after filling;
b) about 2N to about 4.5N after storage at 25° C.±1° C. for 1.5 months after filling;
c) about 2.5N to about 5N after storage at 25° C.±1° C. for 2.5 months after filling; or
d) about 2.5N to about 5.5N after storage at 25° C.±1° C. for 6 months after filling.

19. The method of claim 1, wherein the syringe is provided in a needle shield device comprising a needle sleeve and a plunger; and wherein a manual force can be applied to the plunger to expel the 150 mg/mL omalizumab formulation and unlatch the needle sleeve so that the needle sleeve can cover the needle after injection.

20. A syringe comprising:
a reservoir filled with X mL of a 150 mg/mL omalizumab formulation, wherein X is 2, 1 or 0.5, wherein the X mL of the 150 mg/mL omalizumab formulation has 6000 or fewer particles of diameter >10 μm and/or 600 or fewer particles of diameter >25 μm; and
the syringe further comprising:
a stopper, and
a needle having a length of 12.7 mm, a gauge of 27, a minimum internal diameter of 0.277 mm, and 5 bevels on its distal surface, the syringe being configured to expel the X mL of the omalizumab formulation contained in the reservoir through the needle when a force is applied to the stopper, wherein one of the following options is fulfilled when a constant force of 10N is applied to the stopper:
a) when X is 2, the syringe is configured to expel the omalizumab formulation contained in the reservoir through the needle in less than 16 seconds;
b) when X is 1, the syringe is configured to expel the omalizumab formulation contained in the reservoir through the needle in less than 5 seconds; or
c) when X is 0.5, the syringe is configured to expel the omalizumab formulation contained in the reservoir through the needle in less than 4 seconds.

21. The syringe of claim 20 wherein X is 1, wherein the syringe is configured to expel the X mL of the 150 mg/ml omalizumab formulation through the needle in about 4 seconds to less than 5 seconds when a constant force of 10N is applied to the stopper.

22. The method of claim 1, wherein the 150 mg/ml omalizumab formulation is an aqueous solution of 150 mg/mL antibody with 42.1 mg/mL L-arginine hydrochloride, 1.37 mg/mL L-histidine, 2.34 mg/mL L-histidine hydrochloride monohydrate, and 0.4 mg/mL polysorbate 20.

23. The syringe according to claim 20, wherein the 150 mg/ml omalizumab formulation is an aqueous solution of 150 mg/mL antibody with 42.1 mg/mL L-arginine hydrochloride, 1.37 mg/mL L-histidine, 2.34 mg/mL L-histidine hydrochloride monohydrate, and 0.4 mg/mL polysorbate 20.

24. A method of treating a patient with of one or more of: allergic asthma; food allergy; chronic rhinosinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering X mL of 150 mg/ml omalizumab formulation to the patient, wherein X is 2, and wherein the X mL 150 mg/mL omalizumab formulation is administered subcutaneously by a syringe, the syringe comprising:
a reservoir filled with X mL of 150 mg/mL omalizumab formulation, wherein the X mL 150 mg/mL omalizumab formulation has 6000 or fewer particles of diameter ≥10 μm and/or 600 or fewer particles of diameter ≥25 μm; and the syringe further comprising:
a stopper, and
a needle having a gauge of gauge 25 to gauge 29; and
the syringe being configured to expel the omalizumab formulation contained in the reservoir through the needle when a force is applied to the stopper, wherein when a constant force of 10N is applied to the stopper,
the syringe is configured to expel the omalizumab formulation contained in the reservoir through the needle in less than 16 seconds.

25. The method of claim 24, wherein the syringe is configured to expel the omalizumab formulation contained in the reservoir through the needle in one or more of:
a) less than 8.5 seconds when a constant force of 17N is applied to the stopper; or
b) less than 5 seconds when a constant force of 30N is applied to the stopper.

26. The method of claim 25, wherein the syringe is configured to expel X mL of the omalizumab formulation contained in the reservoir through the needle in one or more of:
a) about 16 seconds to about 14 seconds when a constant force of 10N is applied to the stopper;
b) about 8.5 seconds to about 6 seconds when a constant force of 17N is applied to the stopper; or
c) about 5 seconds to about 4 seconds when a constant force of 30N is applied to the stopper.

27. The method of claim 24, wherein the syringe contains:
a) at least 76% main omalizumab charge variant measured by ion exchange chromatography after being filled with 150 mg/ml omalizumab solution stored at 5° C.±1° C.; or
b) at least 77% main omalizumab charge variant measured by ion exchange chromatography after being filled with 150 mg/mL omalizumab solution and then stored at 5° C.±1° C. for 2.5 months.

28. The method of claim 24, wherein the proportion of the first peak of the syringe measured by hydrophobic interaction chromatography is at least:
a) 62% after filling with 150 mg/mL omalizumab solution stored at 5° C.±1° C.; or
b) 62% after being filled with 150 mg/mL omalizumab solution and then stored at 5° C.±1° C. for 2.5 months.

29. The method of claim 24 wherein the syringe contains at least 0.4 mg/mL polysorbate 20 after filling with 150 mg/mL omalizumab solution stored at 5° C.±1° C.

30. The method of claim 24, wherein the needle has a gauge of gauge 26 to gauge 29.

31. The method of claim 30, wherein when the needle has a gauge of 25, the needle has one of:
a) a minimum internal diameter of 0.292 mm, or
b) a minimum internal diameter of 0.232 mm.

32. The method of claim 30, wherein when the needle has a gauge of 26, the needle has one of:
a) a minimum internal diameter of 0.292 mm; or
b) a minimum internal diameter of 0.232 mm.

33. The method of claim 30, wherein when the needle has a gauge of 27, the needle has one of:
a) a minimum internal diameter of 0.277 mm;
b) a minimum internal diameter of 0.191 mm;
c) a minimum internal diameter of 0.184 mm; or
d) a minimum internal diameter of 0.241 mm.

34. The method of claim 30, wherein when the needle has a gauge of 28, the needle has one of:
a) a minimum internal diameter of 0.133 mm; or
b) a minimum internal diameter of 0.190 mm.

35. The method of claim 30, wherein when the needle has a gauge of 29, the needle has one of:
a) a minimum internal diameter of 0.265 mm;
b) a minimum internal diameter of 0.240 mm;
c) a minimum internal diameter of 0.190 mm; or
d) a minimum internal diameter of 0.133 mm.

36. The method of claim 24 wherein the stopper has a fluoro-resin on the product contacting side or an ethylene tetrafluoroethylene (ETFE) barrier film lamination.

37. The method of claim 24, wherein the stopper has a B2-40 UV cured lubrication coating or is lubricated with 1000 cSt silicone oil.

38. The method of claim 24, wherein the syringe has an internal coating of 0.7±0.2 mg of polydimethylsiloxane.

39. The method of claim 24, wherein the needle has 3 bevels on its distal surface or 5 bevels on its distal surface.

40. The method of claim 24, wherein the apparent viscosity of the 150 mg/mL omalizumab formulation is 12 to 14 mPa·s or, when measured at a shear rate of 200 s⁻¹ is: 24.7±0.5 mPa·s at 5.0±0.2° C., 15.3±0.5 mPa·s at 15.0±0.2° C., 11.4±0.5 m·Pas at 25.0±0.2° C., or 7.2±1.2 mPa·s at 40.0±0.2° C.

41. The method of claim 24, wherein the syringe has a latex-free needle shield.

42. The method of claim 24, wherein the syringe has a round flange at a proximal end of the reservoir; optionally wherein the round flange has a maximum diameter of 11±0.25 mm or 14.7±0.25 mm.

43. The method of claim 24, wherein when the stopper is not inserted into the reservoir, it has one of:
a) a maximum external diameter of 6.67 mm to 7.10 mm and/or a length of 7.85±0.4 mm; or
b) a maximum external diameter of 6.70±0.15 mm and/or a length of 7.85±0.4 mm.

44. The method of claim 24, wherein the syringe has a length of 54.0±0.5 mm and an internal diameter of 8.65 mm±0.05 mm.

45. The method of claim 24, wherein the stopper has a maximum external diameter 9.05±0.15 mm and/or length 7.70±0.4 mm when not inserted into the reservoir.

46. The method of claim 24, wherein the stopper has at least one circumferential rib of outer diameter 9.00-0.15 mm.

47. The method of claim 24, wherein a break loose force configured to move the stopper of the present invention from an initial stationary point to a velocity of 190 mm/min is one or more of:
a) about 2N to about 3.5N at 25° C.±1° C. after filling;
b) about 2N to about 4.5N after storage at 25° C.±1° C. for 1.5 months after filling;
c) about 2.5N to about 5N after storage at 25° C.±1° C. for 2.5 months after filling;
and/or or d) about 2.5N to about 5.5N after storage at 25° C.±1° C. for 6 months after filling.

48. The method of claim 24, wherein the syringe is provided in a needle shield device comprising a needle sleeve and a plunger; and wherein a manual force can be applied to the plunger to expel the 150 mg/mL omalizumab formulation and unlatch needle sleeve so that the needle sleeve can cover the needle after injection.

49. A syringe comprising:
a reservoir filled with X mL of 150 mg/mL omalizumab formulation, wherein X is 2, wherein the X mL of 150 mg/mL omalizumab formulation has 6000 or fewer particles of diameter ≥10 μm and/or 600 or fewer particles of diameter ≥25 μm; and the syringe further comprising:
a stopper, and
a needle having a gauge of gauge 25 to gauge 29,
the syringe being configured to expel X mL of the omalizumab formulation contained in the reservoir through the needle when a force is applied to the stopper, wherein when a constant force of 10N is applied to the stopper:
the syringe is configured to expel the omalizumab formulation contained in the reservoir through the needle in less than 16 seconds.

50. The syringe of claim 49, wherein the syringe is configured to expel X mL of 150 mg/ml omalizumab formulation through the needle in less than 5 seconds to about 4 seconds when a constant force of 10N is applied to the stopper.

51. The method of claim 24, wherein the 150 mg/ml omalizumab formulation is 150 mg/mL antibody with 42.1 mg/mL L-arginine hydrochloride, 1.37 mg/mL L-histidine, 2.34 mg/mL L-histidine hydrochloride monohydrate, 0.4 mg/mL polysorbate 20 as an aqueous solution.

52. A method of treating a patient with one or more of: allergic asthma; food allergy; chronic rhinosinusitis with nasal polyps; chronic spontaneous urticaria; nasal polyps; moderate to severe persistent asthma in patients with a positive skin test or in vitro reactivity to a perennial aeroallergen and symptoms that are inadequately controlled with inhaled corticosteroids, the method comprising administering 300 mg omalizumab formulation to the patient, wherein the method comprises administering 2 mL of 150 mg/mL omalizumab formulation subcutaneously by one syringe filled with 2 mL 150 mg/mL omalizumab formulation.

53. The syringe according to claim 49, wherein the 150 mg/ml omalizumab formulation is 150 mg/mL antibody with 42.1 mg/mL L-arginine hydrochloride, 1.37 mg/mL L-histidine, 2.34 mg/mL L-histidine hydrochloride monohydrate, 0.4 mg/mL polysorbate 20 as an aqueous solution.
